(19) United States
(12) Patent Application Publication
(10) Pub. No.: US 2023/0293428 A1
(43) Pub. Date: Sep. 21, 2023

(54) Drug delivery via contact lenses and similar ophthalmic devices

(71) Applicant: Colorado School of Mines, Golden, CO (US)

(72) Inventor: Anuj Chauhan, Golden, CO (US)

(21) Appl. No.: 17/990,321

(22) Filed: Nov. 18, 2022

Related U.S. Application Data
(60) Provisional application No. 63/281,616, filed on Nov. 19, 2021.

(51) Int. Cl. A61K 9/00; A61K 47/22; A61K 47/10; A61K 31/5377

(52) U.S. Cl. CPC A61K 9/0051; A61K 47/22; A61K 47/10; A61K 31/5377

(57) ABSTRACT

Methods of loading vitamin E onto contact lenses for use as drug-delivering ophthalmic devices are provided, as are contact lenses made by such methods and methods of treatment employing such contact lenses. The disclosed methods and devices achieve high vitamin E loading rates and significantly extend the duration of release of both hydrophilic and hydrophobic drugs, while limiting swelling of the lenses and risk of damage to the lenses during manufacture.

### CROSS REFERENCE TO RELATED APPLICATION

This application claims priority to U.S. Provisional Patent Application 63/281,616, filed 19 Nov. 2021, the entirety of which is hereby incorporated by reference.

### STATEMENT REGARDING FEDERALLY SPONSORED RESEARCH

This invention was made with government support under grant number 1903704 awarded by the National Science Foundation. The government has certain rights in the invention.

### FIELD

This disclosure relates generally to intraocular drug delivery, and particularly to ophthalmic devices, such as contact lenses, and methods of use thereof that enable controlled, extended, and/or sustained release of a drug into an eye of a subject.

### BACKGROUND

Currently, eye drops are the most used dosage form for delivery of ophthalmic drugs because of their simplicity and ease of use for patients. However, drug delivery via eye drops is suboptimal because the corneal bioavailability of most drugs is very low (generally 1 to 5%) when administered via this route. The low bioavailability has two major causes: (1) eye drops remain in the eye for only a few minutes after application because the volume of a single eye drop (usually about 30 μL) is much greater than the volume of liquid that is typically present as a tear film on the surface of the eye (usually about 7 μL), causing rapid drainage of the eye drops from the eye after application, and (2) even during the short period of contact of the eye drops on the eye surface, a large fraction of the drug tends to be removed via the vascularized conjunctiva rather than being made available to the cornea. Additionally, the maximum drug concentration in eye drops is limited by toxicity concerns, which together with the low bioavailability serves to limit the extent to which the drug can be exposed to target tissue(s) in the eye. This combination of factors generally necessitates the application of eye drops multiple times per day to treat many ophthalmic diseases and conditions, which may result in poor patient compliance.

The limitations of eye drop therapies have driven considerable research in the development of alternative dosage forms and administration methods for sustained release of ophthalmic drugs. Particularly, contact lenses are promising vehicles for drug delivery because they significantly increase the bioavailability of the drug compared to eye drops, although commercially available contact lenses are not optimal for sustained delivery because most hydrophilic drugs are completely released from drug-loaded contact lenses within a few hours. Thus, various approaches for modifying contact lenses to allow for extended drug ophthalmic drug delivery have been developed; these approaches include biomimetic imprinting, incorporation of nanoparticles and/or lipids, layered lenses, and incorporation of vitamin E barriers.

The use of vitamin E barriers in contact lenses adapted for delivery of ophthalmic drugs is beneficial because vitamin E is a powerful antioxidant and can improve the stability of the drug(s) incorporated into the lenses. Loading vitamin E onto contact lenses can also significantly slow the release (and thus extend the duration of release) of both hydrophobic and hydrophilic drugs; the barriers force hydrophilic drug molecules to diffuse from the lens via a more tortuous pathway, and hydrophobic drug molecules are dissolved in the vitamin E at high concentrations, thereby resulting in a depot effect. Another potential advantage of loading vitamin E onto contact lenses for drug delivery is the possibility of tailoring the design to achieve a target release profile by controlling the concentration of vitamin E in the lens. Vitamin E incorporation also does not significantly affect the optical, chemical, or mechanical properties of the lens, such as optical clarity, water content, Young's modulus, etc.

Conventional methods of preparing vitamin E-loaded contact lenses have called for simply soaking the lenses in a solution of vitamin E in ethanol, followed by ethanol extraction in buffer, resulting in “trapping” of vitamin E in the lenses. However, when exposed to ethanol, the polymer matrices of conventional contact lenses swell significantly, making the contact lenses fragile and easily damaged. More recently, some research has been devoted to the application of supercritical carbon dioxide for vitamin E loading in silicone hydrogel; while this enables greater vitamin E loading efficiency, the loading procedure is extremely complex and requires impregnation of carbon dioxide in its supercritical state into the contact lens.

There is thus a need in the art for methods of loading vitamin E onto contact lenses to allow for controlled, extended, and/or sustained release of one or more drugs from the contact lens, without causing swelling of the lenses or requiring the use of complex loading techniques. There is a further need in the art for contact lenses made by these methods, and the use of such contact lenses to treat a disease or disorder in a subject.

### SUMMARY

In an aspect of the present disclosure, a method for modifying a contact lens to form an ophthalmic device suitable for drug delivery comprises (a) providing a solution comprising vitamin E as a solute and a mixture of ethanol and water as a solvent, wherein an ethanol:water volume ratio in the solution is between about 60:40 and about 99:1; (b) placing a contact lens in the solution for a first period; and (c) extracting ethanol from the contact lens by placing the contact lens in an aqueous liquid for a second period.

In embodiments, the ethanol:water volume ratio may be between about 70:30 and about 90:10. The ethanol:water volume ratio may, but need not, be about 75:25 or about 85:15.

In embodiments, at least one of the first and second periods may be between about 1 hour and about 48 hours. The at least one of the first and second periods may, but need not, be about 24 hours.

In embodiments, the method may further comprise loading a drug into the contact lens. The drug may, but need not, be timolol or a pharmaceutically acceptable salt, polymer, acid, or ester thereof.

In another aspect of the present disclosure, a contact lens suitable for ophthalmic delivery of a drug comprises at least about 0.5 wt % vitamin E.

In embodiments, the contact lens may comprise at least about 10 wt % vitamin E. The contact lens may, but need not, comprise at least about 20 wt % vitamin E.

In embodiments, the contact lens may further comprise a hydrophilic drug. The hydrophilic drug may, but need not, be timolol or a pharmaceutically acceptable salt, polymer, acid, or ester thereof. A diffusion coefficient of the hydrophilic drug in the contact lens under ambient conditions may, but need not, be less than about 1·10⁻¹³ m²/s.

In embodiments, the contact lens may further comprise a hydrophobic drug.

In another aspect of the present disclosure, a method for administering a drug to a subject in need thereof comprises applying a contact lens to an eye of the subject, wherein the contact lens comprises the drug and at least about 0.5 wt % vitamin E.

In embodiments, the contact lens may comprise at least about 10 wt % vitamin E.

In embodiments, the drug may be a hydrophilic drug. The hydrophilic drug may, but need not, be timolol or a pharmaceutically acceptable salt, polymer, acid, or ester thereof.

In embodiments, the drug may be a hydrophobic drug.

In embodiments, the applying step may be carried out for at least a duration of release of the drug from the contact lens, and the duration of release may be at least about 5 hours.

In another aspect of the present disclosure, a method for modifying a contact lens to form an ophthalmic device suitable for drug delivery comprises (a) providing a solution comprising vitamin E as a solute and a mixture of ethanol and water as a solvent, wherein an ethanol:water volume ratio in the solution is about 75:25 or about 85:15; (b) placing a contact lens in the solution for a first period; (c) extracting ethanol from the contact lens by placing the contact lens in an aqueous liquid for a second period; and (d) loading timolol or a pharmaceutically acceptable salt, polymer, acid, or ester thereof into the contact lens, wherein at least one of the first and second periods is about 24 hours.

In another aspect of the present disclosure, a contact lens suitable for ophthalmic delivery of a drug comprises at least about 20 wt % vitamin E and a hydrophilic drug, wherein a diffusion coefficient of the hydrophilic drug in the contact lens under ambient conditions is less than about 1·10⁻¹³ m²/s.

In another aspect of the present disclosure, a method for administering a drug to a subject in need thereof comprises applying a contact lens to an eye of the subject, wherein the contact lens comprises the drug and at least about 10 wt % vitamin E, wherein the drug is timolol or a pharmaceutically acceptable salt, polymer, ester, or acid thereof, wherein the applying step is carried out for at least a duration of release of the drug from the contact lens, wherein the duration of release is at least about 5 hours.

While specific embodiments and applications have been illustrated and described, the present disclosure is not limited to the precise configuration and components described herein. Various modifications, changes, and variations which will be apparent to those skilled in the art may be made in the arrangement, operation, and details of the methods and systems disclosed herein without departing from the spirit and scope of the overall disclosure.

As used herein, unless otherwise specified, the terms “about,” “approximately,” etc., when used in relation to numerical limitations or ranges, mean that the recited limitation or range may vary by up to 10%. By way of non-limiting example, “about 750” can mean as little as 675 or as much as 825, or any value therebetween. When used in relation to ratios or relationships between two or more numerical limitations or ranges, the terms “about,” “approximately,” etc. mean that each of the limitations or ranges may vary by up to about 10%; by way of non-limiting example, a statement that two quantities are “approximately equal” can mean that a ratio between the two quantities is as little as 0.9:1.1 or as much as 1.1:0.9 (or any value therebetween), and a statement that a four-way ratio is “about 5:3:1:1” can mean that the first number in the ratio can be any value of at least 4.5 and no more than 5.5, the second number in the ratio can be any value of at least 2.7 and no more than 3.3, and so on.

The embodiments and configurations described herein are neither complete nor exhaustive. As will be appreciated, other embodiments are possible utilizing, alone or in combination, one or more of the features set forth above or described in detail below.

### BRIEF DESCRIPTION OF THE DRAWINGS

FIG. 1 is a graph illustrating vitamin E loading on contact lenses as a function of vitamin E concentration in the loading solution, where the solvent is pure ethanol (square data points, shallowest line at right), 85:15 (v/v) ethanol:water (diamond data points, line at center), or 75:25 (v/v) ethanol:water (circular data points, steepest line at lower left).

FIGS. 2A through 2C are graphs of the transmittance spectra and (in inset) images of vitamin E-loaded contact lenses prepared from vitamin E in pure ethanol solvent (FIG. 2A), vitamin E in 85:15 (v/v) ethanol:water solvent (FIG. 2B), and vitamin E in 75:25 (v/v) ethanol:water solvent (FIG. 2C), respectively. Inset images labeled (a) are “blank” (unloaded) lenses, and inset images (b) through (d) are images of lenses loaded with increasing amounts of vitamin E.

FIGS. 3A through 3D are cross-sectional scanning electron microscopy (SEM) of a blank (unloaded) contact lens (FIG. 3A), a 19.1 wt % vitamin E-loaded lens prepared in a pure ethanol solvent (FIG. 3B), a 19.9 wt % vitamin E-loaded lens prepared in an 85:15 (v/v) ethanol:water solvent (FIG. 3C), and a 7.6 wt % vitamin E-loaded lens prepared in a 75:25 (v/v) ethanol:water solvent, respectively.

FIG. 4A is a graph illustrating dynamic vitamin E uptake in contact lenses over time from solutions of vitamin E in 75:25 (v/v) ethanol:water loading solvents, where the concentration of vitamin E in each loading solution was 0.1 wt % (circular data points), 0.2 wt % (square data points), or 0.3 wt % (diamond data points).

FIG. 4B is a graph illustrating vitamin E uptake in contact lenses as a function of vitamin E concentration in the loading solution, as measured by weight difference (circular data points, steeper trend line) and spectral difference (square data points, flatter trend line).

FIG. 5 is a graph illustrating timolol release from timolol-loaded contact lenses over time.

### DETAILED DESCRIPTION

Unless defined otherwise, all technical and scientific terms used herein have the same meaning as is commonly understood by one of ordinary skill in the art. All patents, applications, published applications, and other publications to which reference is made herein are incorporated by reference in their entirety. If there is a plurality of definitions for a term herein, the definition provided in the Summary prevails unless otherwise stated.

As used herein, unless otherwise specified, the terms “administering” and “administration” include administration methods in which a drug is directly administered to a subject, e.g., by putting the drug directly into a dosage form that is administered to the subject, as well as methods in which the drug is indirectly administered to a subject, e.g., by putting a precursor or prodrug of the drug directly into a dosage form that is administered to the subject.

As used herein, the term “ophthalmic device” refers to a device or a composition of matter that is adapted or configured to be placed in physical contact with the cornea. “Ophthalmic devices,” as that term is used herein, may be provided in the form of one or more contact lenses.

As used herein, the term “pharmaceutical composition” refers to a composition of matter comprising at least one active pharmaceutical ingredient that is adapted or configured to be administered to an animal for a therapeutic purpose.

As used herein, the term “subject” refers generally to an animal, including but not limited to a human, to which a composition, device, or formulation provided by the present disclosure is administered or is to be administered. Other animals that may be “subjects” as those terms are used herein include but are not limited to companion animals, such as cats, dogs, and horses; livestock animals, such as cattle, goats, sheep, and pigs; mice; and rats.

Unless otherwise specified, all references herein to any drug encompass, in addition to the base drug, any and all pharmaceutically acceptable salts, polymers, esters, and acids thereof.

Embodiments of the present disclosure generally include an ophthalmic device comprising a drug. Embodiments of the present disclosure also include methods of administration of such devices to a subject, which in some embodiments is a human, to treat a disease or condition and/or achieve a physiological objective. The compositions and methods exhibit advantageous efficiency and health benefits as compared to prior art compositions, devices, and methods. The compositions, devices, and methods are generally provided to treat, prevent, or reduce the risk of a disease or disorder, including but not necessarily limited to an ophthalmic disease or disorder.

Pharmaceutical compositions provided by the present disclosure may be formulated in a unit dosage form. A unit dosage form refers to a physically discrete unit suitable as a unitary dose for subjects undergoing treatment, with each unit containing a predetermined quantity of the active compound calculated to produce an intended therapeutic effect. A unit dosage form may be for a single daily dose, for administration 2 times per day, or one of multiple daily doses, e.g., 3 or more times per day. When multiple daily doses are used, a unit dosage form may be the same or different for each dose. One or more dosage forms may comprise a dose, which may be administered to a subject at a single point in time or during a time interval.

A dose may be administered in a single dosage form or in multiple dosage forms. When multiple dosage forms are used the amount of compound contained within each dosage form may be the same or different. The amount of active compound contained in a dose may depend on the route of administration and whether the disease in a subject is effectively treated by acute, chronic, or a combination of acute and chronic administration.

A significant advantage of the present disclosure is that the total dosage of the drug may be reduced relative to that needed to achieve similar results using eye drops or ophthalmic devices of the prior art. As a result, compositions and methods of the present disclosure may be effective to treat or prevent a disease or condition, and/or achieve a physiological objective, in dosages less than those needed to achieve the same results with prior art compositions, devices, and methods.

In the methods of the treatment disclosed herein, the dosage may be varied during the course of treatment. In embodiments, two or more discrete dosage steps may be used, wherein a first dosage may be administered to the subject for a first period and a second dosage, which may be higher or lower than the first dosage, may be administered to the subject for a second period. By way of non-limiting example, each of the first and second periods may be at least about one day, or at least about two days, or at least about three days, or at least about four days, or at least about five days, or at least about six days, or at least about one week, or at least about two weeks, or at least about three weeks, or at least about one month, or at least about two months, or at least about three months, or at least about four months, or at least about five months, or at least about six months, or at least about seven months, or at least about eight months, or at least about nine months, or at least about ten months, or at least about eleven months, or at least about one year. In embodiments, the dosage may also be continually ramped (i.e. gradually increased) or tapered (i.e. gradually decreased). The use of two or more distinct dosages, or of a ramped or tapered dosing regimen, may be beneficial where, by way of non-limiting example, it is desired to treat two or more diseases or conditions, simultaneously and/or sequentially, or where the severity of a disease or condition to be treated may vary over time.

Ophthalmic devices of the present disclosure may be provided in any suitable form and physical manifestation. By way of non-limiting example, the ophthalmic devices can be administered to a subject in the form of a contact lens. Ophthalmic devices of the present disclosure may thus comprise any suitable pharmaceutically acceptable additives, binders, and/or fillers, and may also comprise multiple (i.e. two or more) therapeutic agents.

In some embodiments of the present disclosure, ophthalmic devices may be, or may be adapted to be, placed or held on the cornea of a subject for a predetermined length of time before being removed. Such embodiments may particularly include those embodiments in which the ophthalmic device is provided in the form of a contact lens. By way of non-limiting example, the length of time may be from about 1 hour to about 30 days, or alternatively in any range having a lower bound of any whole number of hours from about 1 hour to about 720 hours and an upper bound of any whole number of hours from about 1 hour to about 720 hours. In some embodiments, the length of time may be about 1 hour, about 2 hours, about 3 hours, about 4 hours, about 5 hours, about 6 hours, about 7 hours, about 8 hours, about 9 hours, about 10 hours, about 11 hours, about 12 hours, about 13 hours, about 14 hours, about 15 hours, about 16 hours, about 17 hours, bout 18 hours, about 19 hours, about 20 hours, about 21 hours, about 22 hours, about 23 hours, about 1 day, about 2 days, about 3 days, about 4 days, about 5 days, about 6 days, about 7 days, about 8 days, about 9 days, about 10 days, about 11 days, about 12 days, about 13 days, about 14 days, about 15 days, about 16 days, about 17 days, about 18 days, about 19 days, about 20 days, about 21 days, about 22 days, about 23 days, about 24 days, about 25 days, about 26 days, about 27 days, about 28 days, about 29 days, or about 30 days, or any length in any range bounded by any two of these values.

The present inventor has found that administration of a drug to a subject in need thereof by the compositions, devices, and methods of the present disclosure may treat a disease or condition and/or achieve a physiological objective in the subject more effectively than the compositions, devices, and methods of the prior art. Specifically, the cost, inconvenience, and risk of side effects to the subject may be reduced.

Preferred dosages and treatment lengths for the methods of the present disclosure may vary according to the particular disease or condition to be treated and/or the particular physiological objective to be achieved. By way of non-limiting example, administration of compositions and devices according to the present disclosure may be continued for an indefinite period of time, e.g. as a maintenance regimen, for the treatment or prevention of a chronic condition, or for continual or continuous maintenance of one or more tissues or organs, including but not necessarily limited to the eye generally and/or the cornea specifically, in a functional and/or healthy condition. By way of further non-limiting example, administration of compositions and devices according to the present disclosure may be discontinued upon resolving an acute condition or upon achieving a physiological objective of treatment.

The present inventor has investigated transport of vitamin E from ethanol/water solutions to determine the solubility of vitamin E in these solutions and the equilibrium uptake of vitamin E onto contact lenses placed in the solutions, which depends on the partition coefficient of vitamin E. The results of these investigations show that the partition coefficient of vitamin E increases more than 5-fold and more than 10-fold when the water content in the loading solution reaches 15 vol % and 25 vol %, respectively, compared to a pure ethanol solvent. The solubility of vitamin E in the solutions decreases as water fraction increases, but the present inventor has unexpectedly and advantageously found that the increase in partition coefficient outweighs this drawback and allows for loading of at least as much as 20 wt % vitamin E into the lens. Scanning electron microscopy (SEM) characterization also reveals the additional advantage that the vitamin E barriers maintain their morphology on the contact lenses regardless of the water content in the loading solution.

The present inventor has also investigated the release profile of a hydrophilic drug (timolol) from drug- and vitamin E-loaded contact lenses made according to the present disclosure. The release profiles and diffusion coefficients are broadly similar for all lenses with similar vitamin E loads. The present inventor therefore concludes that the devices and methods of the present disclosure have the unexpected advantage that the inclusion of water in the loading solutions promotes vitamin E loading efficiency while maintaining the morphology of vitamin E barriers on the lenses and allowing for extended release of hydrophilic drugs. Additionally, the reduced swelling of the lenses during vitamin E loading is beneficial in that it minimizes the possibility of damage to the lens during the loading step.

Methods for manufacturing a contact lens according to the present disclosure include a step of placing a contact lens in a solution comprising vitamin E as a solute and a mixture of ethanol and water as a solvent for a first period to load vitamin E onto or into the contact lens. The ethanol/water mixture includes a volume fraction of water from about 1 vol % to about 40 vol %, or alternatively in any range having a lower bound of any whole number of volume percent from about 1 vol % to about 40 vol % and an upper bound of any whole number of volume percent from about 1 vol % to about 40 vol %; in other words, an ethanol:water volume fraction of the ethanol/water mixture may be from about 60:40 to about 99:1, or alternatively in any range having a lower bound of any whole-number ratio from about 60:40 to about 99:1 and an upper bound of any whole-number ratio from about 60:40 to about 99:1. The first period is generally selected to allow loading of vitamin E into or onto the contact lens to reach equilibrium with the surrounding solution, which in embodiments may be between about 1 hour and about 48 hours and in particular embodiments may be about 24 hours.

Methods for manufacturing a contact lens according to the present disclosure include a step of extracting ethanol from the vitamin E-loaded contact lens by placing the contact lens in an aqueous liquid for a second period. The second period is generally selected to allow substantially all of the ethanol to be extracted from the contact lens, which in embodiments may be between about 1 hour and about 48 hours and in particular embodiments may be about 24 hours.

Contact lenses and similar ophthalmic devices according to the present disclosure, and/or made by methods according to the present disclosure, may include vitamin E in any amount from about 0.1 wt % to about 50 wt %, or alternatively in any range having a lower bound of any whole number of tenths of a weight percent from about 0.1 wt % to about 50 wt % and an upper bound of any whole number of tenths of a weight percent from about 0.1 wt % to about 50 wt %. In some embodiments, a contact lens may include vitamin E in an amount of about 0.5 wt %, about 1 wt %, about 1.5 wt %, about 2 wt %, about 2.5 wt %, about 3 wt %, about 3.5 wt %, about 4 wt %, about 4.5 wt %, about 5 wt %, about 5.5 wt %, about 6 wt %, about 6.5 wt %, about 7 wt %, about 7.5 wt %, about 8 wt %, about 8.5 wt %, about 9 wt %, about 9.5 wt %, about 10 wt %, about 10.5 wt %, about 11 wt %, about 11.5 wt %, about 12 wt %, about 13 wt %, about 13.5 wt %, about 14 wt %, about 14.5 wt %, about 15 wt %, about 15.5 wt %, about 16 wt %, about 16.5 wt %, about 17 wt %, about 17.5 wt %, about 18 wt %, about 18.5 wt %, about 19 wt %, about 19.5 wt %, about 20 wt %, about 20.5 wt %, about 21 wt %, about 21.5 wt %, about 22 wt %, about 22.5 wt %, about 23 wt %, about 23.5 wt %, about 24 wt %, about 24.5 wt %, or about 25 wt %, or any amount in any range bounded by any two of these values. As described throughout this disclosure, the vitamin E “load” of the contact lens may be controlled, optimized, selected, and/or tuned to provide for a desired release profile and/or duration of release of a selected drug from the contact lens.

Contact lenses according to the present disclosure may be “daily wear” (DW) contact lenses adapted to be worn for one waking day and removed before sleeping; “extended wear” (EW) contact lenses adapted to be worn continuously during both waking and sleeping hours for up to six consecutive full days; or “continuous wear” (CW) contact lenses adapted to be worn continuously during both waking and sleeping hours for up to 30 consecutive full days. The duration of release of a selected drug from the contact lens may be less than, about equal to, or more than the intended wear cycle of the contact lens.

The disclosure is further described by reference to the following non-limiting examples.

### Example 1

Vitamin E Loading in Contact Lenses

Vitamin E solutions were prepared by dissolving vitamin E ((±)-tocopherol, C₂₉H₅₀O₂, >96% purity, HPLC grade) in pure ethanol (C₂H₆O, ACS/USP grade). In some solutions, no water was added (i.e. the solvent was pure ethanol), while in other solutions water was added until a volume ratio of ethanol to water of 85:15 or 75:25 was achieved. The formulations of the vitamin E solutions prepared are shown in Table 1 below. Vitamin E was loaded onto contact lenses by placing commercially available Acuvue Oasys contact lenses (Johnson & Johnson Vision Care, Inc.) into the vitamin E solutions under ambient conditions until equilibrium was reached (about 24 hours). After this period, the contact lenses were gently withdrawn from the vitamin E solutions and placed in fresh water for 1 minute to cause them to shrink to their original shape and size. The lenses were then dipped in pure ethanol for 5 seconds to dissolve any vitamin E residues on the surfaces of the lenses. The vitamin E-loaded contact lenses were then transferred to excess water solution for an additional 24 hours to extract the remaining ethanol in the lens, then stored in Dulbecco's phosphate buffered saline (PBS) solution without calcium or magnesium for further use and characterization.

TABLE 1
Formulations of vitamin E-in-ethanol/water solutions

| | Ethanol in solvent | Water in solvent | Vit. E in solution |
|---|---|---|---|
| Solution no. | (vol %) | (vol %) | (wt %) |
| 1 | 100 | 0 | 2 |
| 2 | 100 | 0 | 4 |
| 3 | 100 | 0 | 6 |
| 4 | 85 | 15 | 1 |
| 5 | 85 | 15 | 2 |
| 6 | 85 | 15 | 4 |
| 7 | 75 | 25 | 0.1 |
| 8 | 75 | 25 | 0.2 |
| 9 | 75 | 25 | 0.3 |

The diameters of lenses soaked in each of the nine solutions according to this procedure were measured after storage in PBS solution; the results of these measurements are given in Table 2 (compared against swelling of lenses in pure water). It was observed that the degree of swelling decreased with increasing water content in the loading solution, as expected.

Vitamin E loading was calculated based on the ratio of the weight difference to the actual weight of the dehydrated lens after vitamin E incorporation, or in mathematical terms

$$VE ( \% ) = \frac{Δ w}{w} \cdot 100 \% = \frac{w - {w}_{0}}{w} \cdot 100 \%$$

where w is the weight of the dehydrated lens after vitamin E loading and wo is the initial weight of the dehydrated lens. In pure ethanol solutions, vitamin E concentrations of 2, 4, and 6 wt % (solutions 1, 2, and 3) resulted in 8.4, 19.1, and 30.5 wt % loading onto the lenses, respectively. When 15 vol % water was added to the solvent, the vitamin E loading became more effective: 9.3, 19.9, and 44.5 wt % loading from 1, 2, and 4 wt % vitamin E solutions in 85:15 ethanol:water solvent (solutions 4, 5, and 6), respectively. As the water fraction in the solvent increased still further to 25 vol %, solubility of vitamin E in the solutions decreased, but solutions of 0.1, 0.2, and 0.3 wt % vitamin E (solutions 7, 8, and 9) still resulted in 0.5, 4.2, and 8.6 wt % vitamin E loading onto the lenses, respectively.

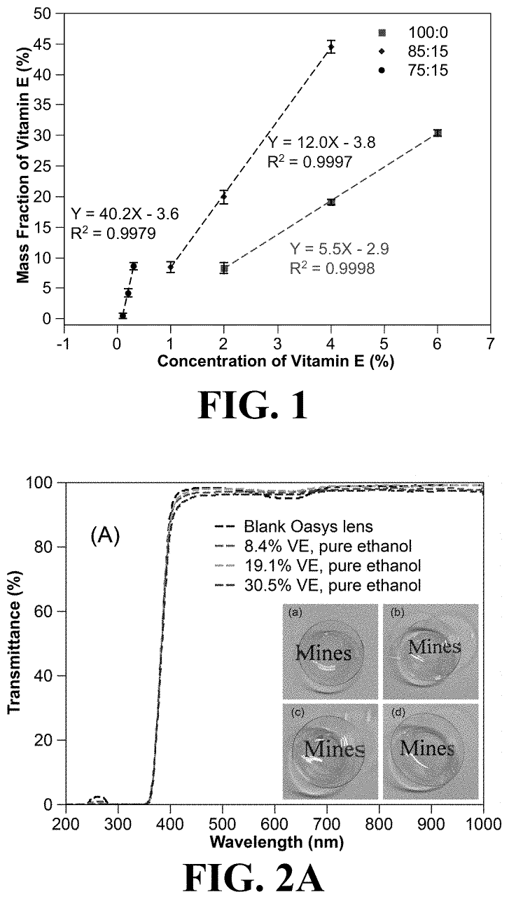

FIG. 1 illustrates the linear relationship between the vitamin E loading in the lens and the vitamin E concentration in the loading solution. The best-fit slope increases with increasing water content in the loading solution, further confirming that inclusion of water promotes vitamin E transport from the solution to the contact lens.

TABLE 2
Average diameter of contact lenses soaked in vitamin E solutions

| Water in solvent (vol %) | Lens diameter (mm) |
|---|---|
| 0 | 21.54 ± 0.29 |
| 15 | 18.26 ± 0.33 |
| 25 | 16.04 ± 0.51 |
| 100 | 13.95 ± 0.16 |

### Example 2

Contact Lens Characterization

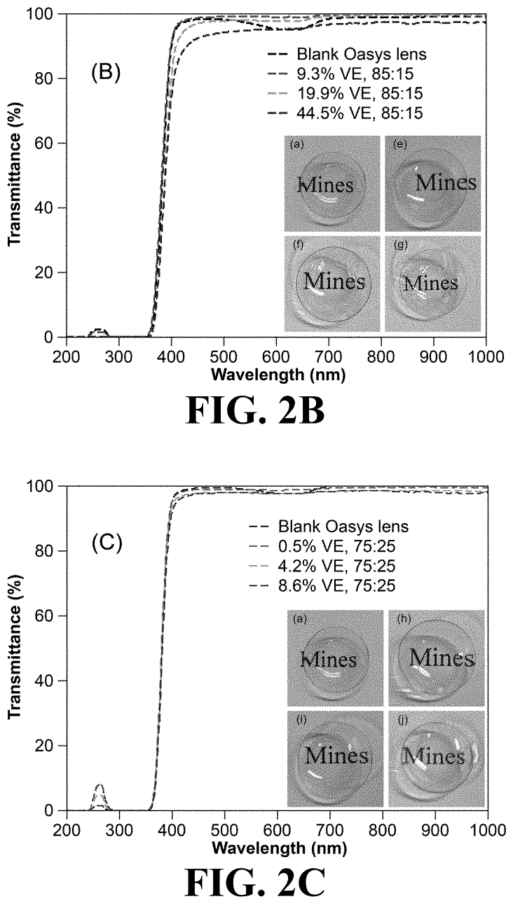

Vitamin E loading in each contact lens was measured based on the weight difference of the dehydrated contact lens after vitamin E loading using a Mettler Toledo AX26 Analytical Balance. The vitamin E loading was also measured by monitoring the dynamic concentration of vitamin E in the loading solution using a Thermo Scientific Genesys™ 150 UV-Vis spectrophotometer in a wavelength range of 245 to 264 nm. The optical transmittance spectra of both “blank” (unloaded”) Acuvue Oasys lenses and vitamin E-loaded lenses were measured using the same spectrophotometer in a wavelength range of 200 to 1000 nm. Referring now to FIGS. 2A through 2C, the transmittance spectra and photographs illustrate that vitamin E-loaded contact lenses remain transparent with greater than 90% transmittance in the visible range of the spectrum.

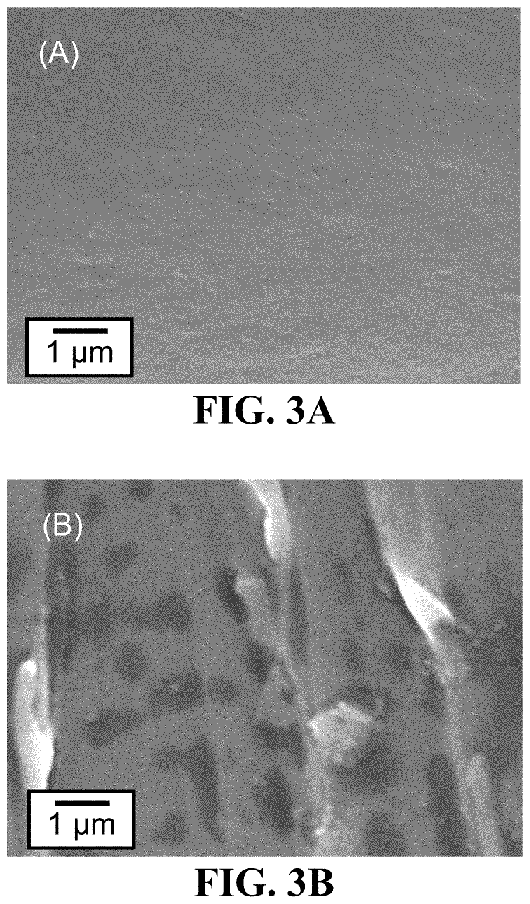

The morphology of the vitamin E barriers inside the contact lenses was characterized by a JEOL 7000 FE scanning electron microscope. Dehydrated contact lenses were cut to expose the cross-section and were attached to the stubs with a vertical sidewall, followed by a thin gold film deposition to increase the electronic conductivity. The SEM images were taken at a working distance of 6 mm under an acceleration voltage of 8 kV. Referring now to FIGS. 3B through 3D, the vitamin E-loaded lenses show vitamin E aggregates about 1 μm in size; SEM imaging involves methods of sample preparation that may impact distribution of the vitamin E barriers, so it is difficult to infer the true sizes of the barriers in the lenses, but the fact that all of the SEM images appear similar suggests that the morphology of the vitamin E barriers in the lens is not strongly affected by the water content of the loading solutions.

### Example 3

Drug Loading in Contact Lenses and Drug Release in Vitro

Timolol maleate was dissolved in PBS to prepare a 1.5 mg/mL solution. Each hydrated contact lens (both blank and vitamin E-loaded) was placed in 3 mL of this solution for 7 days to load the drug in the lenses.

After drug loading, the lenses were removed from the timolol maleate solution and gently wiped with KimWipe to remove the excess solution remaining on the surface. Each lens was then placed in 3 mL of fresh PBS solution for in vitro drug release. The optical absorption spectra of the release media were measured by the same spectrophotometer used in Example 2. The concentration of the timolol in the release medium was determined by fitting the optical absorption spectra in the range of 260 to 340 nm with the calibration spectrum of timolol maleate using MATLAB's minimization of least square error method. The drug release profiles were fitted using a perfect sink model to determine drug diffusivity. By solving the Fick's law diffusion equation, the mass percent of the drug released as a function of time is given by

$$m ( \% ) = 1 - \frac{8}{{π}^{2}} {\sum }_{0}^{∞} \frac{exp ( - \frac{{( 2 n + 1 )}^{2} {π}^{2}}{4 {h}^{2}} Dt )}{{( 2 n + 1 )}^{2}}$$

where h is the half-thickness of the contact lens, D is the diffusivity of the drug molecule in the contact lens, and t is the duration of drug release.

The partition coefficient K of the drug is defined as the ratio of the concentration of the drug in the lens to the concentration of the drug in the medium when equilibrium is reached during the drug loading process. Because the volume of the loading solution is much greater than the volume of the lens, it is assumed that the concentration of the drug in the loading solution remains equal to the known initial concentration. The concentration in the lens after 7 days of loading can be determined by dividing the total mass of the drug in the lens by the volume, and the mass of the drug can be determined from the release data based on the assumption that 100% of the drug is released due to the large volume of release medium compared to the lens volume. Therefore, the partition coefficient K is calculated as

$$K = \frac{{C}_{lens}}{{C}_{loading}} = \frac{{V}_{release} {C}_{release}}{{V}_{lens} {C}_{loading}}$$

where Cₗₑₙₛ and C_{loading} are the concentration of the drug in the lens and the loading solution at equilibrium, respectively, Vᵣₑₗₑₐₛₑ and Vₗₑₙₛ are the volume of the release medium and the contact lens, respectively, and Cᵣₑₗₑₐₛₑ is the final concentration of the drug in the release medium. The calculated partition coefficients are shown in Table 3.

TABLE 3
Partition coefficients of vitamin E, calculated
from weight of dehydrated lenses

| Solution no. | K |
|---|---|
| 1 | 1.15 ± 0.13 |
| 2 | 1.31 ± 0.03 |
| 3 | 1.40 ± 0.02 |
| 4 | 4.32 ± 0.47 |
| 5 | 4.64 ± 0.22 |
| 6 | 5.21 ± 0.10 |
| 7 | 3.40 ± 2.97 |
| 8 | 16.03 ± 2.43 |
| 9 | 23.24 ± 1.38 |

In loading solutions using pure ethanol as the solvent, the partition coefficients ranged from 1.15 to 1.40, which is consistent with previous investigations. When 15 vol % water was introduced into the solvent, the partition coefficient increased to a range of 4.32 to 5.21, and when the water content of the solvent reached 25 vol %, the partition coefficient ranged from 3.40 to 23.24. The partition coefficient is observed to be much more strongly dependent on vitamin E concentration in the 75:25 ethanol:water solutions than in the 85:15 ethanol:water or pure ethanol solutions. Without wishing to be bound by any particular theory, the present inventor hypothesizes that in pure ethanol and 85:15 ethanol:water solutions, vitamin E is present in the swollen contact lenses as either dissolved in the liquid phase within the contact lens material or adsorbed onto the surface of the lens and does not aggregate until the ethanol is extracted by soaking in buffer (whereupon the solubility of vitamin E is reduced, resulting in phase separation and formation of the vitamin E barriers), whereas in the 75:25 ethanol:water solutions, vitamin E begins to form barriers in the lens during loading, i.e. prior to ethanol extraction, resulting in an increasing partition coefficient due formation of the new barrier phase in the lens.

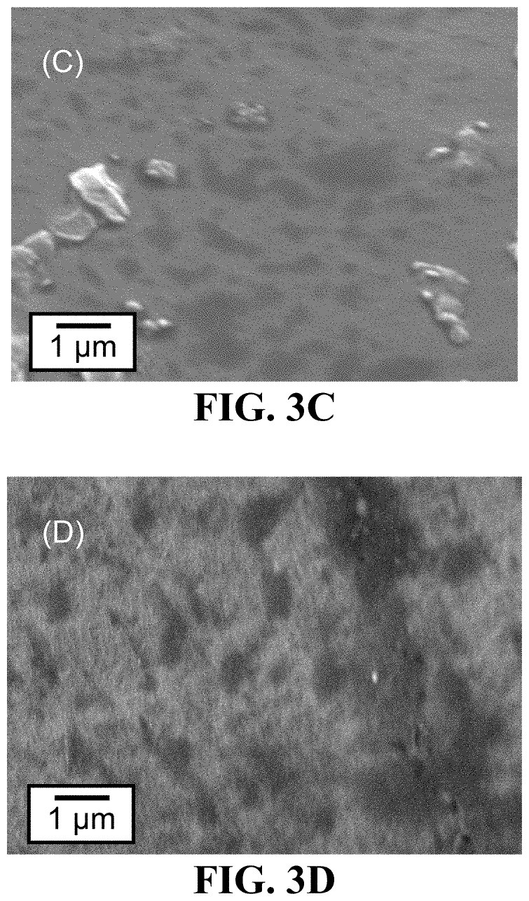

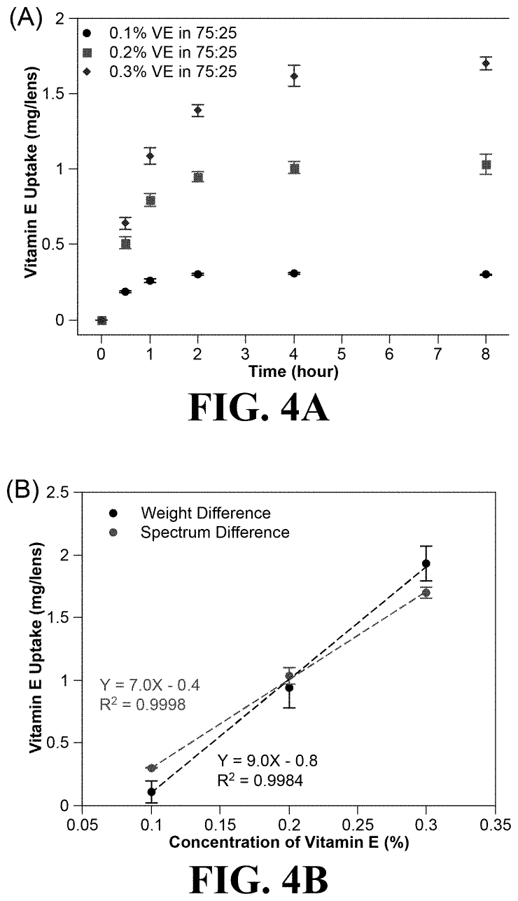

The increase in partition coefficient with increasing vitamin E concentration is a surprising, unexpected, and advantageous result. Therefore, the present inventor developed an additional method to further validate the measurements of vitamin E uptake: monitoring the dynamic absorbance spectrum of vitamin E in the solution over time. It was assumed that the vitamin E loss in the loading solution was equal to the vitamin E uptake in the contact lens due to the mass balance of vitamin E in the system. Referring now to FIG. 4A, equilibrium uptake of vitamin E was reached in about 4 hours in 75:25 ethanol:water solutions. FIG. 4A also shows that the equilibrium vitamin E uptake was also affected by the vitamin E concentration in the loading solution; vitamin E concentrations of 0.1, 0.2, and 0.3 wt % (solutions 7, 8, and 9) resulted in uptakes of 0.3, 1.0, and 1.7 mg/lens, respectively. The results of vitamin E uptake from spectrum analysis are compared against the results from the weight difference measurement in FIG. 4B.

The partition coefficients for lenses loaded in 75:25 ethanol solution were also calculated from vitamin E uptake and are given in Table 4. As Table 4 illustrates, the partition coefficient of vitamin E in solutions 7, 8, and 9 ranged from 10.62 to 19.86, thereby evincing a weakened dependency on concentration compared to the results based on weight measurements; the differences are more pronounced at lower concentrations, likely due to small differences in weight gain that reduce the sensitivity.

TABLE 4
Partition coefficients of vitamin E, calculated by vitamin E uptake

| Solution no. | K |
|---|---|
| 7 | 10.62 ± 0.10 |
| 8 | 17.40 ± 0.98 |
| 9 | 19.86 ± 0.43 |

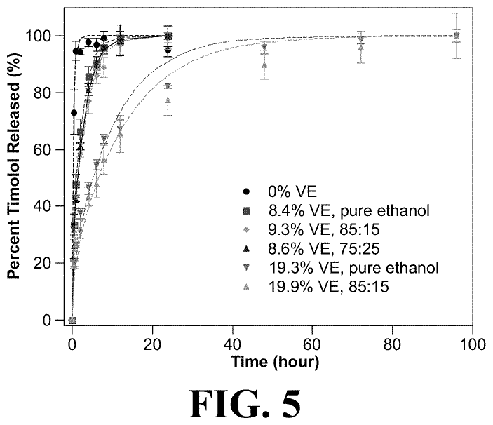

As FIG. 5 illustrates, timolol release lasted around 1 hour from blank (non-vitamin E-loaded) lenses; with vitamin E loaded, the timolol release was significantly extended. To further investigate this effect, the diffusion coefficients of timolol in the lenses were calculated by fitting the release curves to Fick's diffusion equation as described above. The calculated diffusion coefficients D and partition coefficients K are listed in Table 5. First, the release profiles of contact lenses all having vitamin E loads of 8 to 10 wt %, but made from different loading solutions, were compared (represented by the square, diamond, and darker upward-pointing triangular data points in FIG. 5); it was found that in all cases, the release time was extended to about 12 hours and the release profiles were similar. As Table 5 illustrates, the diffusion coefficients of timolol from these lenses are about a third of the diffusion coefficient from control (blank/unloaded) lenses (circular data points in FIG. 5), which results in extension of release by about 10 hours. Two lenses having similar vitamin E loads of 19 to 20 wt % made by two different solutions were also compared (represented by the downward-pointing triangular and lighter upward-pointing triangular data points in FIG. 5); in both cases the timolol release was extended for more than 70 hours, and the release profiles and fitted diffusion coefficients of both lenses were similar. The partition coefficients of timolol in the lenses are found to describe well the loading of timolol in different lenses and it is further found that the partition coefficient is largely independent of the formulation of the contact lenses. Therefore, without wishing to be bound by any particular theory, the present inventor concludes that the method of loading vitamin E does not significantly impact timolol transport, further supporting the conclusion that the method of loading does not significantly affect the morphology of the vitamin E barriers.

TABLE 5
Diffusion and partition coefficients of
timolol maleate from contact lenses

| Vitamin E | | | |
|---|---|---|---|
| wt % | Solvent used | D (m²/s) | K |
| 0 | n/a | 2.04 · 10⁻¹³ ± 0 | 0.7125 ± 0.0168 |
| 8.4 | Pure EtOH | (7.42 ± 1.49) · 10⁻¹⁴ | 0.6050 ± 0.0171 |
| 9.3 | 85:15 EtOH:H₂O | 5.09 · 10⁻¹⁴ ± 0 | 0.7969 ± 0.0242 |
| 8.6 | 75:25 EtOH:H₂O | (6.20 ± 0.329) · 10⁻¹⁴ | 0.6757 ± 0.0152 |
| 19.3 | Pure EtOH | (1.62 ± 0.219) · 10⁻¹⁴ | 0.5739 ± 0.0069 |
| 19.9 | 85:15 EtOH:H₂O | (13.2 ± 0.936) · 10⁻¹⁵ | 0.6108 ± 0.0489 |

The present inventor has measured transport of vitamin E into contact lenses from solutions of vitamin E in ethanol and vitamin E in ethanol/water mixtures, with the goal of loading vitamin E into the lens without significant swelling of the lens. Increasing amounts of water in the loading solution reduce the degree of swelling, which can be beneficial in minimizing the potential for lens damage during manufacture. The solubility of vitamin E decreases with increasing water content in the loading solution, but the partition coefficient increases to compensate for this effect, which allows loading of at least about 20 wt % vitamin E into the lens, sufficient to significantly increase the release duration of a hydrophilic drug (timolol). At vitamin E loads of about 10 wt %, the duration of release of timolol from the lens can be extended from about 1 hour to about 10 hours, and at vitamin E loads of about 20 wt % this can be extended still further to about 70 hours. Based on SEM images and observed effects on timolol diffusivity, without wishing to be bound by any particular theory, it is believed that the morphology of the vitamin E barriers is not significantly impacted by the water fraction in the loading solution. Based on these results, loading vitamin E into contact lenses via solutions of vitamin E in a mixture of water and ethanol has important, surprising, and unexpected advantages relative to loading via solutions of vitamin E in pure ethanol; particularly, comparable vitamin E loads can be achieved while reducing swelling and the potential for lens damage during manufacture.

The concepts illustratively disclosed herein suitably may be practiced in the absence of any element which is not specifically disclosed herein. It is apparent to those skilled in the art, however, that many changes, variations, modifications, other uses, and applications of the disclosure are possible, and changes, variations, modifications, other uses, and applications which do not depart from the spirit and scope of the disclosure are deemed to be covered by the disclosure.

The foregoing discussion has been presented for purposes of illustration and description. The foregoing is not intended to limit the disclosure to the form or forms disclosed herein. In the foregoing Detailed Description, for example, various features are grouped together in one or more embodiments for the purpose of streamlining the disclosure. The features of the embodiments may be combined in alternate embodiments other than those discussed above. This method of disclosure is not to be interpreted as reflecting an intention that the claims require more features than are expressly recited in each claim. Rather, as the following claims reflect, inventive aspects lie in less than all features of a single foregoing disclosed embodiment. Thus, the following claims are hereby incorporated into this Detailed Description, with each claim standing on its own as a separate embodiment.

Moreover, though the present disclosure has included description of one or more embodiments and certain variations and modifications, other variations, combinations, and modifications are within the scope of the disclosure, e.g. as may be within the skill and knowledge of those in the art, after understanding the present disclosure. It is intended to obtain rights which include alternative embodiments to the extent permitted, including alternate, interchangeable, and/or equivalent structures, functions, ranges, or steps to those claimed, regardless of whether such alternate, interchangeable, and/or equivalent structures, functions, ranges, or steps are disclosed herein, and without intending to publicly dedicate any patentable subject matter.

1. A method for modifying a contact lens to form an ophthalmic device suitable for drug delivery, comprising:
(a) providing a solution comprising vitamin E as a solute and a mixture of ethanol and water as a solvent, wherein an ethanol:water volume ratio in the solution is between about 60:40 and about 99:1;
(b) placing a contact lens in the solution for a first period; and
(c) extracting ethanol from the contact lens by placing the contact lens in an aqueous liquid for a second period.

2. The method of claim 1, wherein the ethanol:water volume ratio is between about 70:30 and about 90:10.

3. The method of claim 2, wherein the ethanol:water volume ratio is about 75:25 or about 85:15.

4. The method of claim 1, wherein at least one of the first and second periods is between about 1 hour and about 48 hours.

5. The method of claim 4, wherein the at least one of the first and second periods is about 24 hours.

6. The method of claim 1, further comprising loading a drug into the contact lens.

7. The method of claim 6, wherein the drug is timolol or a pharmaceutically acceptable salt, polymer, acid, or ester thereof.

8. A contact lens suitable for ophthalmic delivery of a drug, comprising at least about 0.5 wt % vitamin E.

9. The contact lens of claim 8, comprising at least about 10 wt % vitamin E.

10. The contact lens of claim 9, comprising at least about 20 wt % vitamin E.

11. The contact lens of claim 8, further comprising a hydrophilic drug.

12. The contact lens of claim 11, wherein the hydrophilic drug is timolol or a pharmaceutically acceptable salt, polymer, acid, or ester thereof.

13. The contact lens of claim 12, wherein a diffusion coefficient of the hydrophilic drug in the contact lens under ambient conditions is less than about 1·10⁻¹³ m²/s.

14. The contact lens of claim 8, further comprising a hydrophobic drug.

15. A method for administering a drug to a subject in need thereof, comprising:
applying a contact lens to an eye of the subject, wherein the contact lens comprises the drug and at least about 0.5 wt % vitamin E.

16. The method of claim 15, wherein the contact lens comprises at least about 10 wt % vitamin E.

17. The method of claim 15, wherein the drug is a hydrophilic drug.

18. The method of claim 17, wherein the hydrophilic drug is timolol or a pharmaceutically acceptable salt, polymer, acid, or ester thereof.

19. The method of claim 15, wherein the drug is a hydrophobic drug.

20. The method of claim 15, wherein the applying step is carried out for at least a duration of release of the drug from the contact lens, wherein the duration of release is at least about 5 hours.

21-23. (canceled)
